(19) United States
(12) Patent Application Publication
(10) Pub. No.: US 2022/0364147 A1
(43) Pub. Date: Nov. 17, 2022

(54) COLOR AND BARDCODED BEADS FOR SINGLE CELL INDEXING

(71) Applicant: Miltenyi Biotec B.V. & Co. KG, Bergisch Gladbach (DE)

(72) Inventors: Olaf HARDT, Bergisch Gladbach (DE); Andreas BOSIO, Bergisch Gladbach (DE); Stefan MILTENYI, Bergisch Gladbach (DE)

(73) Assignee: Miltenyi Biotec B.V. & Co. KG, Bergisch Gladbach (DE)

(21) Appl. No.: 17/771,591

(22) PCT Filed: Nov. 12, 2020
(86) PCT No.: PCT/EP2020/081851

§ 371 Date: Apr. 25, 2022

(30) Foreign Application Priority Data
Nov. 15, 2019 (EP) 19209395.3

(51) Int. Cl. C12Q 1/6806

(52) U.S. Cl. CPC C12Q 1/6806; C12Q 1/6869

(57) ABSTRACT

The present invention is directed to a method for identifying nucleic acids of a target cell from a cell population comprising—isolating at least one target cell from the cell population and at least one color-coded composition comprising a solid particle conjugated to an oligonucleotide into one compartment—lysing the isolated target cells—coupling the nucleic acid molecules of the lysed isolated target cells with the oligonucleotide of the color-coded composition forming a first conjugate—determining the sequence of the first conjugate, thereby identifying the target cell. characterized in that at least one target cell and the at least one color-coded composition are selected to be isolated into one compartment according to at least one pre-selected physical property of the target cell combined with at least one pre-selected physical property of the color-coded composition.

### BACKGROUND

The present invention is directed a method for identifying cDNA, DNA or RNA of target cells from a cell population by single cell indexing using a color-coded composition comprising a solid particle which comprises dyes having different emission sprectra as additional information.

Currently, single cell sequencing gets into the focus of scientists of different research areas, because they are interested in unravelling the cellular heterogeneity of tissues. In addition, an analysis on the single cell level can help to better understand the association of phenotype and function of a cell by deciphering the transcriptome of different subtypes of a given cell population.

During the last years, powerful technologies came into the market that allow the analysis of single cells' transcriptomes on a high throughput scale. Many of these techniques are based on microfluidic separation of one single cell and one single bead into one droplet. In the droplet, bead-specific oligos that are bound to the beads capture the mRNA after cell lysis and become cell-specific barcodes after reverse transcription reaction.

Some of the available technologies also allow to selectively isolate single cells from the cell population, e.g. by laser-detection of antibody-staining and subsequent sorting of marked cells.

However, so far it is not possible to selectively isolate single cells from a heterogeneous cell population on a high-throughput level and to assign the sequencing results of the single cells to one of the different cell types.

In a different field of technology, it is known to identify genetic information obtained from a single cell by conjugating the cell with a polynucleotide as barcode. These methods involve synthesizing a library comprising this barcode which can be sequenced in order to identify a single cell.

The biology and the necessary hardware to isolate cells is for example disclosed in U.S. Pat. Nos. 9,388,456 or 9,695,468. However, this technology is focused on single, isolated cells rather than cells in the context of a complex mixture of cells.

### SUMMARY

It was therefore an object of the invention to provide a conjugate which enables the identification of DNA and RNA molecules of single cells, combined with their phenotype as detectable by binders.

First object of the invention is therefore a method for identifying nucleic acids of a target cell from a cell population comprising
isolating at least one target cell from the cell population and at least one color-coded composition comprising a solid particle conjugated to an oligonucleotide into one compartment
lysing the isolated target cells
coupling the nucleic acid molecules of the lysed isolated target cells with the oligonucleotide of the color-coded composition forming a first conjugate
determining the sequence of the first conjugate, thereby identifying the target cell
characterized in that
at least one target cell and the at least one color-coded composition are selected to be isolated into one compartment according to at least one pre-selected physical property of the target cell combined with at least one pre-selected physical property of the color-coded composition.

The target cells/the cell population and the color-coded compositions are provided as mixture of various subpopulations. The isolation step is conducted by choosing at best one target cell and one color-coded composition according to pre-selected properties into one compartment.

To this end, the pre-selected physical properties of the target cell and the color-coded composition (bead) are used to select and isolate a certain cell subpopulation together with a certain bead population. For example, if the pre-selected physical property of the target cell is the presence of a CD4 marker and pre-selected physical property of the color-coded composition (bead) is blue color, all cells and beads having these properties will be sorted into compartments in a 1:1 ratio. Cells having for example CD5 markers and red beads will not be selected/isolated. It is of course possible to pre-select a plurality of physical properties for both cells and beads as long as a 1:1 relation of the selected properties is maintained. For example it is possible to pre-select 5 different physical properties which enables sorting 5 pairs of cells and beads into compartments in a preferable 1:1 ratio.

The pre-selected physical property of the target cells may be selected from the group consisting of shape, size, granularity, organelle composition, ion composition, sugar composition, lipid composition and protein composition.

If the protein composition is used as pre-selected physical property, at least one intracellular or extracellular protein is marked by fluorescence staining. The term protein composition refers to protein expression and post-translational modification.

The pre-selected physical properties of the color-coded composition are defined by the solid particle and may be selected from the group consisting of size, granularity, charge, magnetic moment, one or more colors and one or more intensities of at least one color.

### BRIEF DESCRIPTION OF THE DRAWINGS

FIG. 1a shows the emission spectra of a solid particle comprising two dyes with different emission spectra and concentration, adopted to discriminate 39 different solid particles. The terms “MACSPlex B1” and “MACSPlex B2” refer to different concentrations of two dyes having different emission spectra. FIG. 1b shows a selection of the resulting solid particles

FIG. 2 shows an exemplary gating scheme to identify 39 distinct bead populations and combining those with target cell populations.

### DETAILED DESCRIPTION

### Target Moiety

The target moiety to be detected with the method of the invention can be on any biological specimen, like tissues slices, cell aggregates, suspension cells, or adherent cells.

### Color-Coded Composition

The color-coded compositions comprise a solid particle conjugated to an oligonucleotide.

In a first variant of the method of the invention, the color-coded composition has a composition according to one of the general formulas (Ia) or (Ib)

X-(P-C-B-BR)n (Ia)

X-(P-B-C-BR)n (Ib)

wherein

X is a solid particle,
P (PCRhandle): oligonucleotide comprising 4 to 30 nucleotide residues
C (color specific barcode): oligonucleotide comprising 1 to 8 nucleotide residues
B (bead specific barcode): oligonucleotide comprising 8 to 30 nucleotide residues
BR (binding region): oligonucleotide comprising 3 to 30 nucleotide residues
n: integer=>1
and wherein P, C, B and BR are bound to each other via same or different oligonucleotide units as spacer comprising each comprising 0 to 30 nucleotide residues.

In another variant of the method, the color-coded composition has a composition according to one of the general formulas (IIa), (IIb), (IIc), (IId), (IIe), (IIe) or (IIf)

X-(P-C-B-U-BR)n (IIa)

X-(P-C-U-B-BR)n (IIb)

X-(P-B-C-U-BR)n (IIc)

X-(P-B-U-C-BR)n (IId)

X-(P-U-B-C-BR)n (IIe)

X-(P-U-C-B-BR)n (IIf)

wherein
X is a solid particle,
P (PCRhandle): oligonucleotide comprising 4 to 30 nucleotide residues
C (color specific barcode): oligonucleotide comprising 1 to 8 nucleotide residues
B (bead specific barcode): oligonucleotide comprising 8 to 30 nucleotide residues
BR (binding region): oligonucleotide comprising 3 to 30 nucleotide residues
U (unique molecular identifier): oligonucleotide comprising 5 to 15 nucleotide residues
n: integer=>1
   and wherein P, C, B, U and BR are bound to each other via same or different oligonucleotide units as spacer comprising each comprising 0 to 30 nucleotide residues.

### Barcode Moiety

In the present application, oligonucleotides C (color specific barcode), B (bead specific barcode) and U (unique molecular identifier) are referred to as “barcode” since they allow identifying a single target by their unique sequence.

The moieties C, B and U carrying a barcode may comprise same of different oligonucleotide sequences with the respective, disclosed number of nucleotide residues. As nucleotide residues, the naturally occurring cytosine (C), adenine (A), guanine (G)and thymine (T) are preferred. By randomly polymerizing these units, a library of oligonucleotides with different sequences can be obtained. For example, a library randomly producing oligonucleotides comprising 10 nucleotide residues will have 4¹⁰=1048576 members.

The oligonucleotide sequences P (PCRhandle), C (color specific barcode), B (bead specific barcode), U (unique molecular identifier) and BR (binding region) are bound to each other either directly or via further oligonucleotide units as spacer unit. The spacer units may by the same or different oligonucleotides comprising each 0 to 30 nucleotide residues. Preferable, the spacer units are non-specific oligonucleotides.

In a preferred embodiment, one or more spacer unit comprise 0 (zero) nucleotide residues, i.e. the oligonucleotides P (PCRhandle), C (color specific barcode), B (bead specific barcode), U (unique molecular identifier) and BR (binding region) are directly bound to each other.

Oligonucleotid P (PCRhandle) may comprise 4 to 30 nucleotide residues and is serving as binding region for primer for subsequent amplification reactions.

Oligonucleotid C (color specific barcode) may comprise 1 to 8 nucleotide residues which allows the identification of the cell or cell type.

Oligonucleotid B (bead specific barcode) may comprise 8 to 30 nucleotide residues and serves as cell specific barcodes allowing to assign sequencing information to the origin cell

Oligonucleotid U (unique molecular identifier) may comprise 5 to 15 nucleotide residues and serves as identifier for each single nucleic acid molecule in the target cell.

Oligonucleotid BR (binding region) may comprise 3 to 30 nucleotide residues and serves as binding region for nucleic acid molecules of interest of the target cell.

The technique to produce oligonucleotides and libraries thereof is well known to a person skilled in the art, as well as the technologies to amplify isolated oligonucleotides to obtain larger amounts thereof. U.S. Pat. No. 9,388,465 summarizes these technologies.

### Solid Particle

The term “solid particle” refers any material which is not or not readily solvable in aqueous systems usually used for cell handling. The term does not necessarily refer to a certain hardness or a composition/material.

Solid particles X as used in the present invention may be manufactured from any material as long as the solvability in aqueous systems is so low that the particle remains observable or detectable during the method of the invention. For example, solid particle X may comprise poly styrene, poly dextran, both optionally chemically modified with reactive groups to bind dyes or oligonucleotids as spacer units or the PCRhandle P. Suitable reactive groups are for example amino or carboxylic groups.

Solid particles useful for the present invention may be prepared with methods known to the skilled person or as described in the literature. For example, they can be prepared by incorporating dyes into pre-formed polymer beads either by swelling of particles in organic solvent mixtures containing dyes either at room temperature (U.S. Pat. No. 6,514,295 B1) or at elevated temperatures (U.S. Pat. No. 7,507,588 B2). A further method involves shifting of phase equilibria due to water addition to force hydrophobic dyes into the polymer phase (U.S. Pat. No. 6,964,747 B2). Solid particles X beads can also be prepared by polymerization of monomer mixtures including dye labeled monomers (J. Am. Chem. Soc. 2004, 126, 21, 6562-6563) or physical entrapment of hydrophobic dyes during particle formation by polymerization (U.S. Pat. No. 5,073,498).

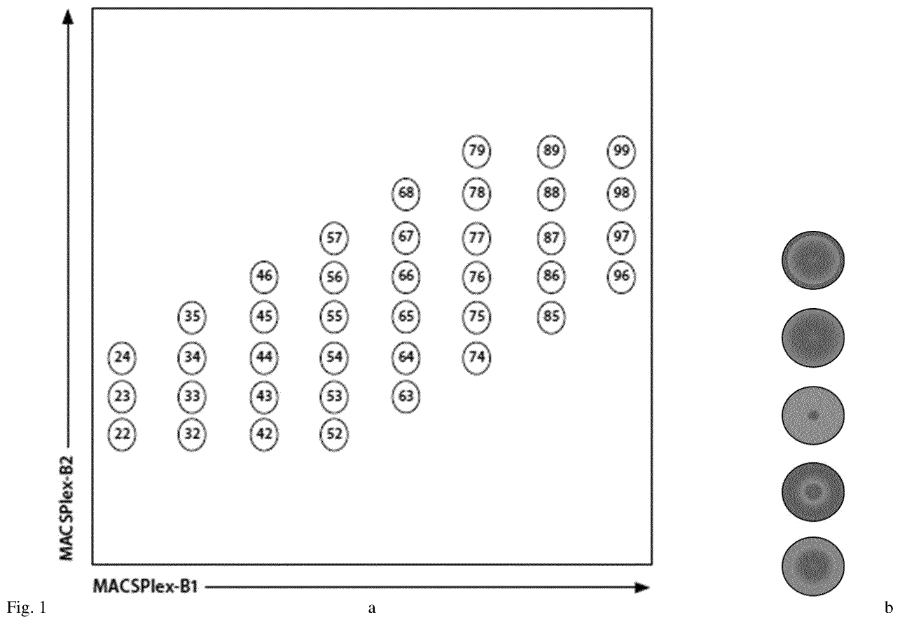

FIG. 1 shows an exemplary layout of a two color bead based wherein B1 and B2 indicate two colors which can be distinguished by a flow cytometer.

In another embodiment of the invention, solid particles may comprise multiple (like 5-50) subunits linked via magnetic force, electrostatic interaction or chemical linkage, which can be covalent or non-covalent. These subunits may be released from each other upon droplet formation, e.g. by chemical or enzymatically induced cleavage.

The size of the solid particle is of minor importance and may be between 1 and 200 μm.

Preferable, the solid particles X comprise at least two dyes having different emission spectra with a difference in emission maxima at least 10 nm, more preferable at least two dyes having different emission spectra with a difference in emission maxima at least 20 nm. While an increasing number of different dyes improves the quality and amount of information, in practical use, 2 to 10 different dyes are sufficient. 1 dye is sufficient in case of only using the concentration as selection criterium.

The concentration and the difference in emission maxima of the dyes are preferable selected in a way that discrimination of at least 30, preferable at least 50 different solid particles is possible.

Useful dyes are for example protein-based, such as phycobiliproteins, polymeric, such as polyfluorenes, small organic molecule dyes, such as xanthenes, like fluorescein, or rhodamines, cyanines, oxazines, coumarins, acridines, oxadiazoles, pyrenes, pyrromethenes, or metallo-organic complexes, such as Ru, Eu, Pt complexes. Besides single molecule entities, clusters of fluorescent proteins or small organic molecule dyes, as well as nanoparticles, such as quantum dots, upconverting nanoparticles, gold nanoparticles, dyed polymer nanoparticles can also be used as fluorescent moieties.

### Method of the Invention

In a further embodiment of the method, the nucleic acids of a target cell to be identified is single-stranded and wherein the complementary strand of the nucleic acid molecule is obtained and coupled to the BR units of the color-coded composition thereby forming a second conjugate and sequencing the second conjugate thereby identifying the target cell.

Single-stranded nucleic acids are for example RNA, denatured DNA or nucleic acid molecules attached to the target cells during the sample preparation procedure. One example for the later are antibody-oligonucleotide conjugates which are used to label the target cells.

The term “determining the sequence of the first/second conjugate” relates to any method known in the art of nucleic acid sequencing and may comprise amplification steps and/or generating a library. In any case, the sequence of the conjugate is obtained, thereby identifying the target cell.

The necessary techniques for coupling strands of nucleic acids with the BR units of the color-coded composition and subsequent sequencing are not of particular relevance for the invention and are known to the person skilled in the art.

In a variant of the methods according to the invention, the at least one target cell is isolated from the cell population with at least one color-coded composition into one compartment by placing the at least one target cell and the at least one color-coded composition into one aqueous droplet surrounded by a fluid immiscible with water.

It is further possible that the target cells belonging to the same cell type or phenotype or cells binding to the same antibodies/analyte are provided with color-coded compositions having the same solid particle X.

In the methods of the invention, the color-coded compositions may be provided with at least 2 different solid particles X in order to provide at least 2 different cell types with color-coded compositions having different solid particles X.

The cell type of the target cells may be identified by sequencing C (color specific barcode) of the conjugate. In a further variant, prior to isolating at least one target cell from the cell population with at least one color-coded composition according to the invention into one compartment, the cell type of the target cell is determined by fluorescence staining.

### Use of the Method

The method of the invention can be used for various applications in research, diagnostics and cell therapy. The method of the invention is especially useful for identifying nucleic acids of a target cell from a cell population. Analytes may be used to identify and measure biomarkers or therapeutic targets.

### EXAMPLES

The following are hypothetical processes according to the method of the invention. The process step of isolating at least one target cell from the cell population with at least one color-coded composition according into one compartment shall be performed on a MACSQuant Tyto machine (obtainable from Miltenyi Biotec B.V. & Co. KG, in the following referred to as “Tyto”) equipped with a MEMS valve positioned in a disposable cartridge capable of placing at least one target cell and at least one color-coded composition into one aqueous droplet surrounded by a fluid immiscible with water. Such valves/cartridges are described in PCT/US 19/27577.

CASE 1: Analysing Gene Expression in Different Subsets of Immune Cells from Blood by Single-Cell-Sequencing.

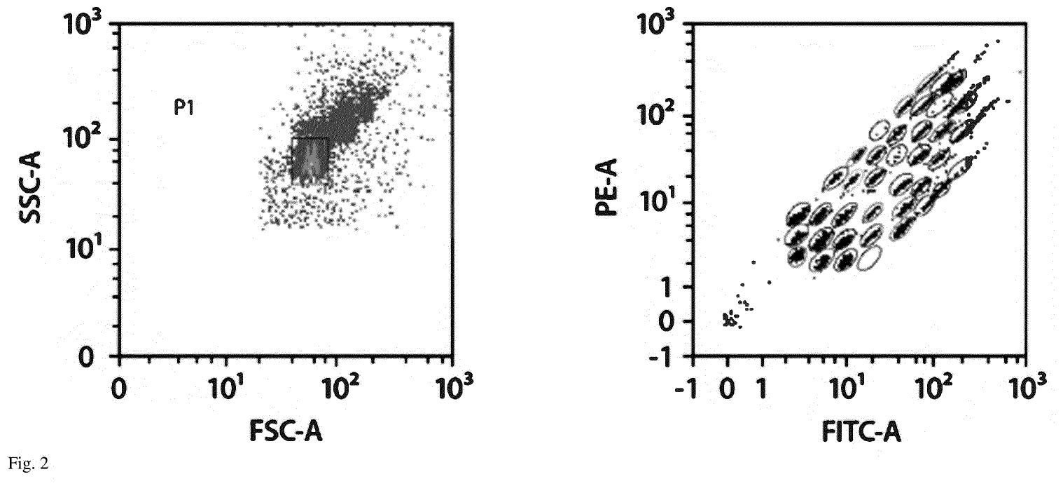

Process Steps
1. Provide cell population like sample preparation of blood (e.g. Ficoll, RBC lysis . . . )
2. Staining the cell population with CD45-VB, CD56-FITC, CD3-PE, CD19-APC, PI (for dead cell exclusion)
3. Mixing stained sample with multiplex beads
4. Loading the sample/bead mixture, oil and lysis buffer into the cartridge, all in separate compartments
5. Initiating sample analysis and manual scatter based gating of the total bead population
6. Tyto automatically defines the 39 fluorescent bead populations;
7. Initiating sample analysis to set sort gates of desired cell populations as follows:
A. Cell gating based on size (scatter) parameters to distinguish cells from 5 μm beads
B. Cell population 1 is Cell gate/Viability gate/CD45+/CD56−/CD3−/CD19+ (B cells)→Set “Sort gate 1”
C. Cell population 2 is Cell gate/Viability gate/CD45+/CD56−/CD3+/CD19− (T cells)→Set “Sort gate 2”
D. Cell population 3 is Cell gate/Viability gate/CD45+/CD56+/CD3−/CD19− (NK cells)→Set “Sort gate 3”
   Exemplary gating results are shown in FIG. 2
8. Tyto automatically combines each sort gate with a fluorescent bead population (e.g. “Sort Gate 1”+“Bead population X”, “Sort Gate 2”+“Bead population Y” . . . ), the pairing information needs to be available for downstream analysis. Example as follows:
→Cell population 1=“Sort gate 1” is paired with GREEN beads (Bead gate/green population gate)
   →Cell population 2=“Sort gate 2” is paired with RED beads (Bead gate/red population gate)
   →Cell population 3=“Sort gate 3” is paired with BLUE beads (Bead gate/blue population gate)
9. Sorting is started, Tyto sorts 5.000 cells of each cell population paired with the respective bead population (as indicated in point 8), each cell-bead doublet is encapsulated into a water-in-oil droplet; cell is first sort event, bead is second event to optimize recovery
10. QC parameters are recorded: number of cells encapsulated, events for each cell population, frequency of correct matching (1 cell and 1 correct bead in droplet), missed target cells, more TBD
11. Cell is automatically lysed in droplet by mixture with lysis buffer, released mRNA is captured by oligonucleotide on bead, droplets are stable
12. Cartridge is removed and incubated for 2 hours at 37° C. to perform reverse transcription leading to cDNA linked to bead specific barcode
13. Water-in-oil droplet are lysed by detergent
14. Bulk barcoded DNA is amplified and sequenced
15. The sequence information is aligned according to the barcodes:
   →Each sequence showing a particular random barcode is coming from one particular cell
   →In addition, each oligonucleotide contains a short barcode specific for one bead population. Thereby, next to knowing which sequences come from a particular cell, the sequence information will also tell which bead population/color this particular cell was paired with, allowing for defining which cell population it initially belonged to, e.g. the particular cell was a Cell gate/Viability gate/CD45+/CD56−/CD3+/CD19− T cell because the sequence belongs to a RED bead

Case 2: Analyze the T Cell Receptor (TCR) Repertoire of Different TIL Subpopulations by Single-Cell-Sequencing

Process Steps:
1. Sample preparation of tumor tissue (GentleMACS based tissue dissociation, filtering and washing)
2. Staining the sample from #1 with CD3-VB, CD4-FITC, CD8-PE, CD25-APC, PI (for dead cell exclusion)
3. Mixing stained sample with multiplex beads
4. Loading the sample/bead mixture, oil and lysis buffer into the cartridge, all in separate compartments
5. Initiating sample analysis and manual scatter based gating of the total bead population
6. Tyto automatically defines the 39 fluorescent bead populations;
7. Initiating sample analysis to set sort gates of desired cell populations:
8. A. Cell gating based on size (scatter) parameters to distinguish cells from 5 μm beads
9. B. Cell population 1 is Cell gate/Viability gate/CD3+/CD4−/CD8+/CD25− (effector T cells)→Set “Sort gate 1”
10. C. Cell population 2 is Cell gate/Viability gate/CD3+/CD4+/CD8−/CD25− (helper T cells)→Set “Sort gate 2”
11. D. Cell population 3 is Cell gate/Viability gate/CD3+/CD4+/CD8−/CD25+ (regulatory T cells)→Set “Sort gate 3”
12. Tyto automatically combines each sort gate with a fluorescent bead population (e.g. “Sort Gate 1”+“Bead population X”, “Sort Gate 2”+“Bead population Y” . . . ), the pairing information has to be available to the customer for downstream analysis. Example as follows
13. →Cell population 1=“Sort gate 1” is paired with GREEN beads (Bead gate/green population gate)
   →Cell population 2=“Sort gate 2” is paired with RED beads (Bead gate/red population gate)
   →Cell population 3=“Sort gate 3” is paired with BLUE beads (Bead gate/blue population gate)
14. Sorting is started, Tyto sorts 10.000 cells of each cell population paired with the respective bead population (as indicated in point 8), each cell-bead doublet is encapsulated into a water-in-oil droplet; cell is first sort event, bead is second event to optimize recovery
15. QC parameters are recorded: number of cells encapsulated, events for each cell population, frequency of correct matching (1 cell and 1 correct bead in droplet), missed target cells, more TBD
16. Cell is automatically lysed in droplet by mixture with lysis buffer, released mRNA is captured by oligonucleotide on bead, droplets are stable
17. Cartridge is removed and incubated for 2 hours at 37° C. to perform reverse transcription leading to cDNA linked to bead specific barcode
18. Water-in-oil droplet are lysed by detergent
19. Bulk barcoded DNA is amplified and sequenced
20. The sequence information is aligned according to the barcodes:
   →Each TCR sequence showing a particular random barcode is coming from one particular cell
   →In addition, each oligonucleotide contains a short barcode specific for one bead population. Thereby, next to knowing which sequences come from a particular cell, the sequence information will also tell which bead population/color this particular cell was paired with, allowing for defining which cell population it initially belonged to, e.g. the particular cell with this specific TCR was a Cell gate/Viability gate/CD3+/CD4−/CD8+/CD25− effector T cell because the sequence belongs to a GREEN bead.

1. A method for identifying nucleic acids of a target cell from a cell population comprising
isolating at least one target cell from the cell population and at least one color-coded composition comprising a solid particle conjugated to an oligonucleotide into one compartment
lysing the isolated target cells
coupling the nucleic acid molecules of the lysed isolated target cells with the oligonucleotide of the color-coded composition forming a first conjugate determining the sequence of the first conjugate, thereby identifying the target cell.
characterized in that
at least one target cell and the at least one color-coded composition are selected to be isolated into one compartment according to at least one pre-selected physical property of the target cell combined with at least one pre-selected physical property of the color-coded composition.

2. The method according to claim 1 characterized in that the pre-selected physical properties of the target cell are selected from the group consisting of shape, size, granularity, organelle composition, ion composition, sugar composition, lipid composition and protein composition.

3. The method according to claim 2 characterized in that protein composition is used as pre-selected physical property wherein at least one intracellular or extracellular protein is marked by fluorescence staining.

4. The method according to claim 1 characterized in that the pre-selected physical properties of the color-coded composition are defined by the solid particle and are selected from the group consisting of size, granularity, charge, magnetic moment, one or more colors and one or more intensities of at least one color.

5. The method according to claim 1 characterized in that the color-coded composition has composition according to one of the general formulas (Ia) or (lb)
X-(P-C-B-BR)n (Ia)
X-(P-B-C-BR)n (lb)
wherein
X is a solid particle,
P (PCRhandle): oligonucleotide comprising 4 to 30 nucleotide residues
C (color specific barcode): oligonucleotide comprising 1 to 8 nucleotide residues
B (bead specific barcode): oligonucleotide comprising 8 to 30 nucleotide residues
BR (binding region): oligonucleotide comprising 3 to 30 nucleotide residues
n: integer=>1
and wherein P, C, B and BR are bound to each other via same or different oligonucleotide units as spacer comprising each comprising O to 30 nucleotide residues.

6. The method according to claim 5 characterized in that the color-coded composition has a composition according to one of the general formulas (Ila), (Ilb), (Ile), (Ild), (Ile), (Ile) or (Ilf)
X-(P-C-B-U-BR)n (Ila)
X-(P-C-U-B-BR)n (Ilb)
X-(P-B-C-U-BR)n (Ile)
X-(P-B-U-C-BR)n (Ild)
X-(P-U-B-C-BR)n (Ile)
X-(P-U-C-B-BR)n (Ilf)
wherein
X is a solid particle,
P (PCRhandle): oligonucleotide comprising 4 to 30 nucleotide residues
C (color specific barcode): oligonucleotide comprising 1 to 8 nucleotide residues
B (bead specific barcode): oligonucleotide comprising 8 to 30 nucleotide residues
BR (binding region): oligonucleotide comprising 3 to 30 nucleotide residues
U (unique molecular identifier): oligonucleotide comprising 5 to 15 nucleotide residues
n: integer=>1
and wherein P, C, B, U and BR are bound to each other via same or different oligonucleotide units as spacer comprising each comprising O to 30 nucleotide residues.

7. The method according to claim 1 characterized in that the at least one target cell is isolated from the cell population with at least one color-coded composition into one compartment by placing the at least one target cell and the at least one color-coded composition into one aqueous droplet surrounded by a fluid immiscible with water.

8. The method according to claim 1 characterized in that the physical property of the target cells used for selection is identified by sequencing C (color specific barcode) of the conjugate.

9. The method according to claim 1 characterized in that the nucleic acids of a target cell to be identified is single-strained and wherein the complementary strand of the nucleic acid molecule is obtained and coupled to the BR units of the color-coded composition thereby forming a second conjugate and sequencing the second conjugate thereby identifying the target cell.
